(19) United States
(12) Patent Application Publication
(10) Pub. No.: US 2014/0234961 A1
(43) Pub. Date: Aug. 21, 2014

(54) SYNTHETIC SINGLE CHAIN VARIABLE DOMAIN (SCFV) IMMUNOGLOBULIN FRAGMENT VEHICLE CONTAINING FUSION PROTEINS FOR TARGETED INTRODUCTION OF siRNA

(71) Applicants: Michael R. Simon, Ann Arbor, MI (US); Reginald Michael Garavito, Williamston, MI (US)

(72) Inventors: Michael R. Simon, Ann Arbor, MI (US); Reginald Michael Garavito, Williamston, MI (US)

(21) Appl. No.: 14/072,500

(22) Filed: Nov. 5, 2013

Related U.S. Application Data
(60) Provisional application No. 61/722,637, filed on Nov. 5, 2012.

(51) Int. Cl. A61K 47/48

(52) U.S. Cl. CPC A61K 47/484; A61K 47/48561
USPC 435/375; 530/387.3

(57) ABSTRACT

A fusion protein and process are provided by which double-stranded RNA containing small interfering RNA nucleotide sequences is introduced into specific cells and tissues. A cell surface receptor specific synthetic single chain variable domain (scFv) immunoglobulin fragment vehicle specific to a cell surface receptor of the cell and having a cell surface receptor specific binding site is provided. An RNA binding protein fused to said scFv is adsorbed with a double-stranded RNA or to a small hairpin RNA sequence complementary to a nucleotide sequence of a target gene in the cell and includes a small interfering RNA operative to suppress production of a target cellular protein. The scFv induces internalization into said cell of the fusion protein subsequent to the binding of said scFV to the cell surface receptor of the target cell.

### RELATED APPLICATIONS

This application is a non-provisional application that claims priority benefit to U.S. Provisional application Ser. No. 61/722,637, filed 5 Nov. 2012, the contents of which are hereby incorporated by reference.

### FIELD OF THE INVENTION

The present invention relates in general to gene product suppression and in particular to gene product suppression through delivery of double-stranded RNA or small hairpin RNA targeting a particular protein within a subject.

### BACKGROUND OF THE INVENTION

RNA interference (RNAi) is the process whereby messenger RNA (mRNA) is degraded by small interfering RNA (siRNA) derived from double-stranded RNA (dsRNA) containing an identical or very similar nucleotide sequence to that of the target gene. (Waterhouse 2001; Hutvagner and Zamore 2002a and 2002b; Lewis 20020132788; Lewis 20030092180; Kreutzer 20040038921; Scaringe 20040058886). This process prevents the production of the protein encoded by the targeted gene. Allele-specific silencing of dominant disease genes can be accomplished (Miller 2003).

The benefits of preventing specific protein production in mammals include the ability to treat disease caused by such proteins. Such diseases include those that are caused directly by such a protein such as multiple myeloma which is caused by harmful concentrations of a monoclonal immunoglobulin as well as diseases in which the protein plays a contributory role such as the effects of inflammatory cytokines in asthma.

Introduction of dsRNA into mammalian cells induces an interferon response which causes a global inhibition of protein synthesis and cell death. However, dsRNA several hundred base pairs in length have been demonstrated to be able to induce specific gene silencing following cellular introduction by a DNA plasmid (Diallo M et al. Oligonucleotides 2003).

### SUMMARY OF THE INVENTION

A fusion protein and process are provided by which double-stranded RNA containing small interfering RNA nucleotide sequences is introduced into specific cells and tissues. A cell surface receptor specific synthetic single chain variable domain (scFv) immunoglobulin fragment vehicle specific to a cell surface receptor of the cell and having a cell surface receptor specific binding site is provided. An RNA binding protein fused to said scFv is adsorbed with a double-stranded RNA or to a small hairpin RNA sequence complementary to a nucleotide sequence of a target gene in the cell and includes a small interfering RNA operative to suppress production of a target cellular protein. The scFv induces internalization into said cell of the fusion protein subsequent to the binding of said scFV to the cell surface receptor of the target cell.

### BRIEF DESCRIPTION OF THE DRAWING

FIGS. 1A and 1B are schematics of an inventive OKT10 scFv protein fusion. In FIG. 1A, a 3D view of the single-chain Fv (scFv) portion juxtaposed against the ectodomain of CD38 (OKT10 epitope highlighted in magenta). In FIG. 1B, the general design of the OKT10 scFv-based siRNA vehicle scFv-Prm1.

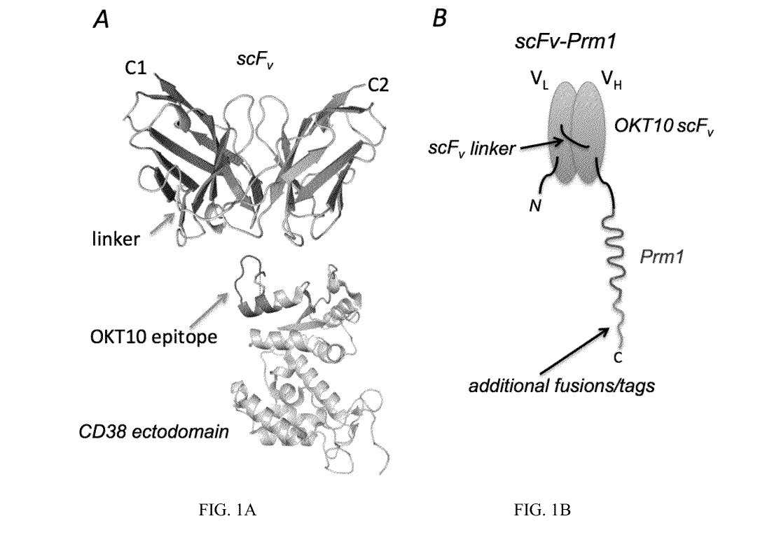

FIG. 2 is a schematic of an inventive OKT10 scFv protein fusion to create multi- and polyvalent siRNA vehicles. On the left is the scFv-Prm1-p73tet fusion, which creates a tetramer displaying 4 scFv-Prm1 domains. On the right, the scFv-Prm1-Fth1 fusion assembles into an oligomer with 24 monomers, which can display 24 scFv-Prm1 domains. The arrows show where some of the scFv-Prm1 domains would be located.

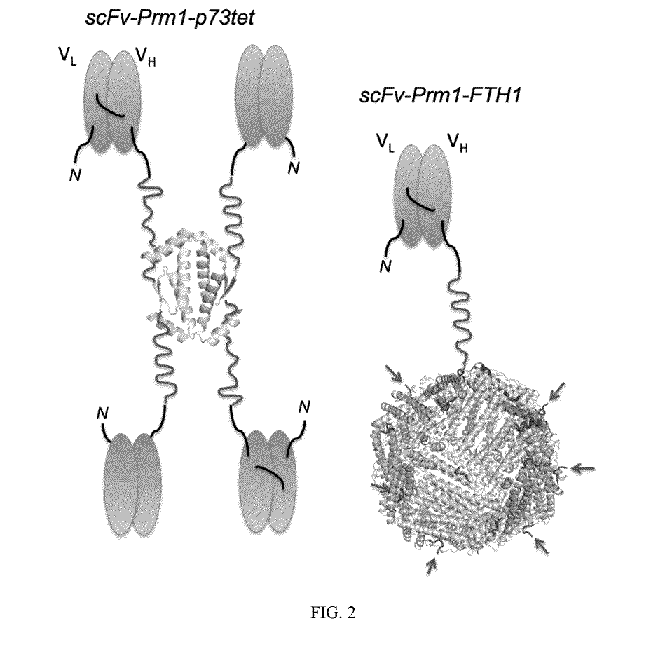

### DETAILED DESCRIPTION OF THE INVENTION

The present invention has utility in suppression of deleterious gene expression products. Production of specific proteins is associated with allergic reactions, transplant organ rejection, cancer, and IgA neuropathy, to name but a few of the medical conditions a subject may suffer. Additionally, according to the present invention, it is appreciated that specific animal proteins are also suppressed in foodstuffs such as cow's milk, through the treatment of the animal. Inventive compositions include one of a long or short dsRNA, or short hairpin RNA (shRNA) that is adsorbed to a RNA binding protein that is integrated into a scFv that includes a cell surface receptor specific ligand such that the RNA binding protein and ligand create a single protein. The ligand is targeted to a specific tissue and/or cell type upon delivery to a subject. In designing a ligand coupled dsRNA or shRNA binding protein, a target tissue and/or cell is selected, and the targeted cell type is analyzed for receptors that internalize ligands following receptor-ligand binding. It is appreciated that the present invention is also operative in suppressing genes within a cell growing in vitro and particularly well suited for limiting contaminants in recombinant protein manufacture.

Cell specific antigens which are not naturally internalized are operative herein by incorporating an arginine-rich peptide within the ligand, an arginine-rich peptide attached to the cell surface receptor specific ligand, as detailed in U.S. Pat. No. 6,692,935 B1 or U.S. Pat. No. 6,294,353 B1. An arginine-rich peptide causes cellular internalization of a coupled molecule upon contact of the arginine-rich peptide with the cell membrane. Pentratin and transportan are appreciated to also be operative as vectors to induce cellular internalization of a coupled molecule through attachment to the cell surface receptor specific ligand as detailed in U.S. Pat. No. 6,692,935 B1 or U.S. Pat. No. 6,294,353 B1.

A cell surface receptor specific ligand as used herein is defined as a molecule that binds to a receptor or cell surface antigen.

The functional RNA interference activity of interfering RNA transported into target cells while adsorbed to a fusion protein containing protamine as the RNA bonding protein and a Fab fragment specific for the HIV envelope protein gp160 has been demonstrated (Song et al. 2005). Similarly, functional RNA interference activity of interfering RNA transported into target cells as a cargo molecule attached to HIV-1 transactivator of transcription (TAT) peptide₄₇₋₅₇ has been demonstrated (Chiu Y-L et al. 2004). The functional RNA interference activity of interfering RNA transported into target cells as a cargo molecule attached to pentratin has also been demonstrated (Muratovska and Eccles 2004).

The dsRNA or shRNA oligonucleotide mediating RNA interference is delivered into the cell by internalization of the receptor.

DsRNA with siRNA sequences that are complementary to the nucleotide sequence of the target gene are prepared. The siRNA nucleotide sequence is obtained from the siRNA Selection Program, Whitehead Institute for Biomedical Research, Massachusetts Institute of Technology, Cambridge, Mass. (http://jura.wi.mit.edu) after supplying the Accession Number or GI number from the National Center for Biotechnology Information website (www.ncbi.nlm.nih.gov). The Genome Database (www.gdb.org) provides the nucleic acid sequence link which is used as the National Center for Biotechnology Information accession number. Preparation of RNA to order is commercially available (Ambion Inc., Austin, Tex.; GenoMechanix, LLC, Gainesville, Fla.; and others). Determination of the appropriate sequences would be accomplished using the USPHS, NIH genetic sequence data bank. Alternatively, dsRNA containing appropriate siRNA sequences is ascertained using the strategy of Miyagishi and Taira (2003). DsRNA may be up to 800 base pairs long (Diallo M et al. 2003). The dsRNA optionally has a short hairpin structure (US Patent Application Publication 2004/0058886). Commercially available RNAi designer algorithms also exist (Life Technologies, Grand Island, N.Y., USA).

Ligand-RNA binding fusion proteins are prepared using existing plasmid technology (Caron et al. 2004; He et al. 2004). RNA binding proteins illustratively include histone (Jacobs and Imani 1988), RDE-4 (Tabara et al. 2002; Parrish and Fire 2001), and protamine (Warrant and Kim 1978). RNA binding protein cDNA is determined using the Gene Bank database (www.ncbi.nlm.nih.gov/IEB/Research/Acembly). For example, RDE-4 cDNA Gene Bank accession numbers are AY07926 and y1L832c2.3. RDE-4 initiates RNA interference by presenting dsRNA to Dicer (Tabara et al).

Additional dsRNA binding proteins (and their Accession numbers in parenthesis) include: PKR (AAA36409, AAA61926, Q03963), TRBP (P97473, AAA36765), PACT (AAC25672, AAA49947, NP_{—}609646), Staufen (AAD17531, AAF98119, AAD17529, P25159), NFAR1 (AF167569), NFAR2 (AF167570, AAF31446, AAC71052, AAA19960, AAA19961, AAG22859), SPNR (AAK20832, AAF59924, A57284), RHA (CAA71668, AAC05725, AAF57297), NREBP (AAK07692, AAF23120, AAF54409, T33856), kanadaptin (AAK29177, AAB88191, AAF55582, NP_{—}499172, NP_{—}198700, BAB19354), HYL1 (NP_{—}563850), hyponastic leaves (CAC05659, BAB00641), ADAR1 (AAB97118, P55266, AAK16102, AAB51687, AF051275), ADAR2 P78563, P51400, AAK17102, AAF63702), ADAR3 (AAF78094, AAB41862, AAF76894), TENR (XP_{—}059592, CAA59168), RNaseIII (AAF80558, AAF59169, Z81070Q02555/S55784, P05797), and Dicer (BAA78691, AF408401, AAF56056, 544849, AAF03534, Q9884), RDE-4 (AY071926), F1120399 (NP_{—}060273, BAB26260), CG1434 (AAF48360, EAA12065, CAA21662), CG13139 (XP_{—}059208, XP_{—}143416, XP_{—}110450, AAF52926, EEA14824), DGCRK6 (BAB83032, XP_{—}110167) CG1800 (AAF57175, EAA08039), F1120036 (AAH22270, XP_{—}134159), MRP-L45 (BAB14234, XP_{—}129893), CG2109 (AAF52025), CG12493 (NP_{—}647927), CG10630 (AAF50777), CG17686 (AAD50502), T22A3.5 (CAB03384) and nameless Accession number EAA14308 as enumerated in Saunders and Barber 2003.

Alternatively, cell surface receptor specific ligands that are rich in arginine and tyrosine residues are constructed such that those residues are positioned to form hydrogen bonds with engineered RNA containing appropriately positioned guanine and uracil (Jones 2001). Additionally, the necessity and performance of an internalization moiety is determined in vitro.

The suitability of the resulting ligand-dsRNA as a substrate for Dicer is first determined in vitro using recombinant Dicer (Zhang H 2002, Provost 2002, Myers J W 2003). Optimal ligand molecule size and dsRNA length are thereby identified.

In one embodiment, the ligand-dsRNA binding molecule(s) illustratively include: a histone (Jacobs and Imani 1988), RDE-4 (Tabara et al. 2002; Parrish and Fire 2001), and protamine (Warrant and Kim 1978) in order to render the ligand-dsRNA hydrophilic. The histone with relatively lower RNA-histone binding affinity (Jacobs and Imani 1988) such as histone H1 (prepared as described by Kratzmeier M et al. 2000) is preferred. Alternatively, RDE-4 is used as prepared commercially (Qiagen, Valencia, Calif.) using RDE-4 cDNA (Gene Bank accession numbers AY07926 and y1L832c2.3). RDE-4 initiates RNA interference by presenting dsRNA to Dicer (Tabara et al).

Protamines are arginine-rich proteins. For example, protamine 1 contains 10 arginine residues between amino acid residue number 21 and residue number 35 (RSRRRRRRSCQTRRR) (Lee et al. 1987) (SEQ ID NO. 1). Protamine binds to RNA (Warrant and Kim 1978).

Preparation of the ligand-histone-dsRNA complex is accomplished as described by (Yoshikawa et al. 2001). Complexes of ligand-lysine rich histone, the histone containing 24.7% (w/w) lysine and 1.9% arginine (w/w), with dsRNA is prepared by gentle dilution from a 2 M NaCl solution. Ligand-histone and dsRNA are dissolved in 2 M NaCl/10 mM Tris/HCl, pH 7.4, in which the charge ratio of dsRNA:histone (−/+) is adjusted to 1.0. Then the 2 M NaCl solution is slowly dispersed in distilled water in a glass vessel to obtain 0.2 M and 50 mM NaCl solutions. The final volume is 200 μL and final dsRNA concentration is 0.75 μM in nucleotide units.

Preparation of the ligand-RDE-4-dsRNA-complex is accomplished as described by (Johnston et al. 1992), for the conserved double-stranded RNA binding domain which RDE-4 contains. Ligand-RDE-4 binding to dsRNA to is accomplished in 50 mM NaCl/10 mM MgCl₂/10 mM Hepes, pH 8/0.1 mM EDTA/1 mM dithiothreitol/2.5% (wt/vol) non-fat dry milk.

Preparation of the ligand-protamine-dsRNA complex is accomplished as described by (Warrant and Kim 1978). The ligand-protamine (human recombinant protamine 1, Abnova Corporation, Taiwan, www.abnova.com.tw) and dsRNA at a molar ratio of 1:4 are placed in a buffered solution containing 40 mM Na cacodylate, 40 mM MgCl₂, 3 mM spermine HCl at pH 6.0 (Warrant and Kim 1978). The solution is incubated at 4° C.-6° C. for several days. Alternatively, the ligand-protamine-dsRNA complex is prepared as described by Song et al. 2005. The siRNA (300 nM) is mixed with the ligand-protamine protein at a molar ratio of 6:1 in phosphate buffered saline for 30 minutes at 4° C.

The constructed ligand-RNA binding protein-dsRNA complex is then administered parenterally and binds to its target cell via its receptor. The constructed ligand-RNA binding protein-dsRNA complex is then internalized and the dsRNA is hydrolyzed by Dicer thereby releasing siRNA for gene silencing.

A therapeutic protein operative in certain embodiments of the present invention is a mutant form of a native protein. Mutants operative herein illustratively include amino acid substitutions relative to amino acid sequences detailed herein. It is further appreciated that mutation of the conserved amino acid at any particular site is preferably mutated to glycine or alanine. It is further appreciated that mutation to any neutrally charged, charged, hydrophobic, hydrophilic, synthetic, non-natural, non-human, or other amino acid is similarly operable.

Modifications and changes are optionally made in the structure (primary, secondary, or tertiary) of the therapeutic protein which are encompassed within the inventive compound that may or may not result in a molecule having similar characteristics to the exemplary polypeptides disclosed herein. It is appreciated that changes in conserved amino acid bases are most likely to impact the activity of the resultant protein. However, it is further appreciated that changes in amino acids operable for receptor interaction, resistance or promotion of protein degradation, intracellular or extracellular trafficking, secretion, protein-protein interaction, post-translational modification such as glycosylation, phosphorylation, sulfation, and the like, may result in increased or decreased activity of an inventive compound while retaining some ability to alter or maintain a physiological activity. Certain amino acid substitutions for other amino acids in a sequence are known to occur without appreciable loss of activity.

In making such changes, the hydropathic index of amino acids are considered. According to the present invention, certain amino acids can be substituted for other amino acids having a similar hydropathic index and still result in a polypeptide with similar biological activity. Each amino acid is assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Without intending to be limited to a particular theory, it is believed that the relative hydropathic character of the amino acid determines the secondary structure of the resultant polypeptide, which in turn defines the interaction of the polypeptide with other molecules. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

The present invention is further detailed with respect to the following non-limiting examples. These examples are not intended to limit the scope of the appended claims.

### Example 1

The Invitrogen Corporation (Carlsbad, Calif.) CellSensor CRE-bla Jurkat Cell-based Assay is used. The detailed protocol is available online and is included in the references (CellSensor protocol). Jurkat cells express CD38 on their cell surfaces which is internalized following ligand binding to it (Funaro at al. 1998). CellSensor CRE-bla Jurkat Cell-based Assay contains a beta-lactamase reporter gene under control of a cAMP response element which has been stably integrated into the CRE-bla Jurkat cell line (clone E6-1). Beta-lactamase is expressed following forskolin stimulation.

Short interfering RNA 19 base pairs long is prepared using the Invitrogen Corporation algorithm based on the DNA sequence of the CRE-bla beta-lactamase gene:

(SEQ ID NO. 2)
ATGGACCCAGAAACGCTGGTGAAAGTAAAAGATGCTGAAGATCAGTTGG
GTGCACGAGTGGGTTACATCGAAC
TGGATCTCAACAGCGGTAAGATCCTTGAGAGTTTTCGCCCCGAAGAACGT
TTTCCAATGATGAGCACTTTTAAAGTTCTGCTATGTGGCGCGGTATTATC
CCGTATTGACGCCGGGCAAGAGCAACTCGGTCGCCGCATACACTATTCTC
AGAATGACTTGGTTGAGTACTCACCAGTCACAGAAAAGCATCTTACGGAT
GGCATGACAGTAAGAGAATTATGCAGTGCTGCCATAACCATGAGTGATAA
CACTGCGGCCAACTTACTTCTGACAACGATCGGAGGACCGAAGGAGCTAA
CCGCTTTTTTGCACAACATGGGGGATCATGTAACTCGCCTTGATCGTTGG
GAACCGGAGCTGAATGAAGCCATACCAAACGACGAGCGTGACACCACGAT
GCCTGTAGCAATGGCAACAACGTTGCGCAAACTATTAACTGGCGAACTAC
TTACTCTAGCTTCCCGGCAACAATTAATAGACTGGATGGAGGCGGATAAA
GTTGCAGGACCACTTCTGCGCTCGGCCCTTCCGGCTGGCTGGTTTATTGC
TGATAAATCTGGAGCCGGTGAGCGTGGGTCTCGCGGTATCATTGCAGCAC
TGGGGCCAGATGGTAAGCCCTCCCGTATCGTAGTTATCTACACGACGGGG
AGTCAGGCAACTATGGATGAACGAAATAGACAGATCGCTGAGATAGGTGC
CTCACTGATTAAGCATTGGTAA.

The DNA nucleotide sequence derived for suppressing beta-lactamase synthesis is: CCACGATGCCTGTAGCAAT (SEQ ID NO. 3). The complementary RNA oligonucleotide is prepared and annealed to its complementary strand sequences. This duplex siRNA is then incubated with anti-CD38 (Fab′)² fragment-histone (RNA binding protein) (Yoshikawa et al. 2001) or anti-CD38 (Fab′)² fragment-protamine (RNA binding protein) (Song et al. 2005). The siRNA-histone or protamine-anti-CD38 complex is incubated at 37° C. with the Jurkat cells for from 4 to 24 hours at concentrations ranging from 100 pM to 200 nM to evaluate efficacy. Typical efficacy is at 2 nM. Effective knockdown of intracellular synthesis of beta-lactamase is demonstrated in this system by the appearance of green cellular fluorescence. Positive control cells, which produce beta-lactamase, fluoresce blue.

### Example 2

Multiple myeloma is a fatal incurable disease caused by the production of large amounts of a monoclonal immunoglobulin by malignant plasma cells (Grethlein S, Multiple Myeloma, eMedicine 2003). CD38 is a cell surface receptor found on myeloma plasma cells (Almeida J et al. 1999). Ligation of CD38 with anti-CD38 monoclonal antibodies (Serotec, Raleigh, N.C. and others) results in CD38 internalization (Pfister et al. 2001).

Anti-CD38 monoclonal antibodies are hydrolyzed by pepsin to produce anti-CD38 (Fab′)² fragments. Histone or protamine-anti CD38 (Fab′)² conjugate is prepared as described by Hermanson (Hermanson 1996, pp 456-493). The histone or protamine-anti-CD38 (Fab′)² conjugate is adsorbed to dsRNA containing a siRNA sequence that is complementary to a portion of the nucleotide sequence of the rearranged heavy chain of IgG (Yoshikawa et al. 2001, Song et al. 2005). In this case the nucleotide sequence link is X98954 and the GI number is 1495616. The siRNA sequences provided by the Whitehead Institute are:

Sense 5′:
(SEQ ID NO. 4)
CGCCAAGAACUUGGUCUAU UU
Antisense 3′:
(SEQ ID NO. 5)
UU GCGGUUCUUGAACCAGAUA.

Alternatively, the histone or protamine-anti-CD38 (Fab′)² conjugate is adsorbed to the dsRNA containing a siRNA sequence that is complementary to a portion of the nucleotide sequence of the rearranged heavy chain of the IgG subclass of the subject's monoclonal IgG, i.e., IgG₁, IgG₂, IgG₃ or IgG₄.

The siRNA is then incorporated into dsRNA. Varying doses ranging from 0.4 to 15 grams of the histone or protamine-anti-CD38 (Fab′)² conjugate dsRNA are administered depending upon response. Effective doses of histone or protamine-anti-CD38 (Fab′)² conjugate dsRNA need to be administered at intervals ranging from one day to several days in order to maintain suppression of IgG production. Because the half life of IgG is up to approximately 23 days, the circulating concentration of the myeloma IgG will decrease gradually over several months. Suppression of the IgG subclass to which the IgG myeloma protein belongs will allow maintenance of IgG mediated immunity because the remaining IgG subclasses are not reduced. Improvement and/or prevention aspects of the disease which are consequences of high concentrations of the myeloma protein occur gradually as the concentration of the myeloma protein decreases. A direct effect of high concentrations of myeloma protein is hyperviscosity. This morbid effect of multiple myeloma is inhibited.

The histone or protamine-anti-CD38 (Fab′)² conjugate dsRNA containing the above described siRNA then binds to CD38 on the surfaces of the subject's plasma cells. Following internalization, Dicer hydrolyzes the dsRNA into siRNA which then interrupts the malignant plasma cell production of IgG myeloma protein.

### Example 3

Allergic disease is mediated via IgE binding to the surfaces of mast cells and basophils. Upon bridging of adjacent IgE molecules by antigen, the mast cells and basophils are activated and release their mediators (Siraganian 1998). IgE binding by mast cells and basophils causes the signs and symptoms of allergic rhinitis, asthma, food and drug allergy, and anaphylaxis (e.g. Becker 2004). The amino acid sequence of the CH3 region of human IgE is available as are many of the codons (Kabat E A 1991). The DNA nucleotide sequence of the CH3 region of human IgE is readily deduced. The deduced CH3 region sequence is then provided to the Whitehead Institute's internet site as above to yield the corresponding siRNA sequence.

The histone or protamine-anti-CD38 (Fab′)² conjugate adsorbed to the anti-IgE siRNA then binds to CD38 on the surfaces of the subject's plasma cells. Following internalization, Dicer hydrolyzes the long dsRNA into siRNA which then interrupts the plasma cell production of the IgE. Over several months, the mast cell-bound and basophil-bound IgE is released and metabolized. The mast cell and basophil IgE receptors decrease markedly and the subject loses allergic reactivity.

### Example 4

IgA nephropathy is an incurable disease of the kidney caused by deposition of IgA in the glomeruli of the kidneys (Brake M 2003). IgA₁ or IgA₂ production is interrupted, depending upon the IgA subclass in the glomeruli, as described above for the silencing of IgG production. The progressive kidney damage caused by IgA is thereby interrupted.

### Example 5

CD177 is a GPI linked cell surface glycoprotein which is expressed on granulocytes and bone marrow progenitor cells such as erythroblasts and megakaryocytes. One of the alleles of CD177 is called PRV-1 and is highly expressed in polycythemia rubra vera (Temerinac S., et al., 2000). CD177 is internalized into the cell when it is bound by antibody (Bauer et al 2007). Antibody to CD177 is available from Biolegend, San Diego, Calif. (cat#315802). There is an activating mutation in the tyrosine kinase Janus kinase 2 (JAK2) in polycythemia vera, essential thrombocythemia, and myeloid metaplasia with myelofibrosis (Scott et al 2007). This mutation is the substitution of phenylalanine for valine at position 617 of the JAK2 gene. The amino acid sequences of the wild type gene and the mutated gene are published (Scott et al 2007). The DNA nucleotide sequence of the wild type and mutated JAK2 genes are readily deduced. The deduced mutated JAK2 gene nucleotide sequence is then provided to the Whitehead Institute's internet site as above to yield the corresponding siRNA sequence. siRNA sequences specific for mutant exon 12 alleles described by Scott et al. 2007 are also generated and used in a composition to specifically target cells expressing JAK2 with an activating mutation.

The histone or protamine-anti-CD177 (Fab′)² [human anti-CD177(Fab′)²] conjugate adsorbed to the anti-JAK2 siRNA then binds to CD177 on the surfaces of the subject's erythroblasts. Following internalization, Dicer hydrolyzes the long dsRNA into siRNA which then interrupts the erythroblast production of the JAK2 kinase. The mutated erythroblasts no longer proliferate and decrease markedly. The subject no longer expresses polycythemia and the disease does not progress to myelofibrosis. Healthy cells which express the wild type JAK2 kinase are not effected and proliferate normally. Essential thrombocythemia, myeloid metaplasia and myelofibrosis are similarly treated.

### Example 6

Design of the single chain variable chain immunoglobulin vehicle fragment (scFv). The cDNA sequences for the variable light (Vₗ) and heavy (Vₕ) chains of the OKT10 mouse monoclonal antibody are obtained from the NCBI Genbank database: (OKT10 Vh chain: ACCESSION ABA42888, OKT10 Vl chain: ACCESSION ABA42887). The cDNA sequences for variable domains of the light and heavy chains (V_{L} and V_{H}, respectively) are joined by a DNA sequence coding for a 14-amino acid linker sequence (-GGGGSGGGSGGGGS-) (SEQ ID No. 6), creating a coding sequence for a “V_{L}-linker-V_{H}” scFv. The cDNA sequence is optimized for codon usage in E. coli K-12, and the resulting cDNA sequence is flanked by the DNA restriction sites NdeI (5′) and BamHI (3′). The final cDNA sequence is synthesized by Life Technologies (Carlsbad, Calif., USA), and coded for an scFv that is 243 amino acids in length (˜26,260 KD). An alternative “V_{H}-linker-V_{L}” scFv vehicle is made by merely reversing the order of linkage between the light and heavy variable chains. The scFv vehicle cDNA (SEQ. ID NO. 7) and amino acid sequence for the scFv vehicle (SEQ. ID NO. 8) are provided below, where the NdeI and BamHI restriction sites are in bold.

(SEQ. ID NO. 7)
CATATGGCCGATATTGTTATGACCCAGAGCCAGAAAATCATGCCGACCA
GCGTTGGTGATCGTGTTAGCGTTACCTGTAAAGCAAGCCAGAATGTTGAT
ACCAATGTTGCATGGTATCAGCAGAAACCGGGTCAGAGCCCGAAAGCAC
TGATTTATAGCGCAAGCTATCGTTATAGCGGTGTTCCGGATCGTTTTACC
GGTAGCGGTAGCGGCACCGATTTTACCCTGACCATTACCAATGTGCAGAG
CGAAGATCTGGCAGAATATTTCTGTCAGCAGTATGATAGTTATCCGCTGA
CCTTTGGTGCAGGTACAAAACTGGATCTGAAACGCGGTGGTGGTGGTTCA
GGTGGTGGTAGCAGTGGTGGCGGTGGTAGCGAAGTTAAACTGATTGAAGC
AGGCGGTGGTCTGGTGCAGCCAGGTGGTAGCCTGAAACTGAGCTGTGCAG
CAAGCGGTTTTGATTTTAGCCGTAGCTGGATGAATTGGGTTCGTCAGGCA
CCGGGTAAAGGTCTGGAATGGATTGGTGAAATTAATCCGGATAGCAGCAC
CATTAACTATACCACCAGTCTGAAAGACAAATTTATCATCAGCCGTGACA
ATGCCAAAAACACCCTGTATCTGCAAATGACCAAAGTTCGTAGCGAAGAT
ACCGCACTGTATTATTGTGCACGTTATGGTAATTGGTTTCCGTATTGGGG
TCAGGGCACCCTGGTTACCGTTAGCGCAGGATCC
(SEQ. ID NO. 8)
MADIVMTQSQKIMPTSVGDRVSVTCKASQNVDTNVAWYQQKPGQSPKALI
YSASYRYSGVPDRFTGSGSGTDFTLTITNVQSEDLAEYFCQQYDSYPLTF
GAGTKLDLKRGGGGSGGGSSSGGGGSEVKLIEAGGGLVQPGGSLKLSCAA
SGFDFSRSWMNWVRQAPGKGLEWIGEINPDSTINYTTSLKDKFIISRDNA
KNTLYLQMTKVRSEDTALYYCARYGNWFPYWGQGTLVTVSAGS

### Example 7

Design of the scFv-fusion. Creating the scFv-anti-CD38 fusion with full length cysteine-free human protamine 1. The amino acid sequence for human protamine 1 is obtained from the NCBI Genbank database (Accession AAA63249) and has the sequence M A R Y R C C R S Q S R S R Y Y R Q R Q R S R R R R R R S C Q T R R R A M R C C R P R Y R P R C R R H (SEQ. ID NO. 9). The native sequence of SEQ. ID NO. 9 is modified to replace all cysteine residues with serine in order to eliminate the possibility of non-specific disulfide bridge formation with the resulting amino acid sequence G S A R Y R S S R S Q S R S R Y Y R Q R Q R S R R R R R R S S Q T R R R A M R S S R P R Y R P R S R R H (SEQ. ID NO. 10), which also includes the residues glycine and serine at the N-terminus due to the BamHI restriction site added to the 5′ end of the cDNA sequence. A predicted cDNA sequence, optimized for codon usage in E. coli K-12, was created, was then synthesized by Life Technologies (Carlsbad, Calif., USA); the recombinant gene sequence codes for a cysteine-free human protamine 1 variant that is 52 amino acids in length. The BamHI DNA restriction site at the 5′ end allows for simple ligation to the 3′ end of the cDNA of the scFv vehicle. This creates the scFv-anti-CD38 fusion with the full length cysteine-free human protamine 1.

Creating multivalent scFv-anti-CD38 fusions with full length cysteine-free human protamine 1 of SEQ ID NO. 9 or SEQ ID NO. 10. A fusion cDNA construct was designed to fuse human protamine 1 (PRM1) with the heavy chain of human ferritin (FTH1); the amino acid sequence for FTH1 was obtained from the NCBI Genbank database (Accession EAW74001.1; GI:119594407) with a length of 183 residues as follows:

(SEQ ID NO: 11)
MTTASTSQVRQNYHQDSEAAINRQINLELYASYVYLSMSYYFDRDDVALK
NFAKYFLHQSHEEREHAEKLMKLQNQRGGRIFLQDIQKPDCDDWESGLNA
MECALHLEKNVNQSLLELHKLATDKNDPHLCDFIETHYLNEQVKAIKELG
DHVTNLRKMGAPESGLAEYLFDKHTLGDSDNES

The resulting amino acid sequence also included the residues glycine and serine at the N-terminus due to the BamHI restriction site added to the 5′ end of the cDNA sequence; an ochre stop signal, followed by XhoI restriction site added to the 3′ end of the cDNA sequence. A predicted cDNA sequence, optimized for codon usage in E. coli K-12, was created, was then synthesized by Life Technologies (Carlsbad, Calif., USA); the final recombinant gene sequence codes for a cysteine-free PRM1-FTH1 fusion that is 238 amino acids in length. The BamHI DNA restriction site at the 5′ end allows for simple ligation to the 3′ end of the cDNA of the scFv vehicle to create the final fusion.

The cysteine-free PRM1-FTH1 fusion that is 238 amino acids in length has the amino acid sequence for human PRM1-FTH1 fusion of: GSARYRSSRSQSRSRY YRQRQRSRRRRRRSSQTRRRAMRSSRPRYRPRSRRHKLGSTTASTSQVRQNY HQDSEAAINRQINLELYASYVYLSMSYYFDRDDVALKNFAKYFLHQSHEERE HAEKLMKLQNQRGGRIFLQDIQKPDCDDWESGLNAMECALHLDKNVNQSLL ELHKLATDKNDPHLCDFIETHYLNEQVKAIKELGDHVTNLRKMGAPESGLAE YLFDKHTLGDSDNES (SEQ ID NO. 12).

Amino acid sequence for human Prm1-Fth1 fusion (linkers are in bold).

SEQ ID NO. 13)
GSARYRSSRSQSRSRYYRQRQRSRRRRRRSSQTRRRAMRSSRPRYRPR
SRRHKLGSTTASTSQVRQNYHQDSEAAINRQINLELYASYVYLSMSYYFD
RDDVALKNFAKYFLHQSHEEREHAEKLMKLQNQRGGRIFLQDIQKPDCDD
WESGLNAMECALHLDKNVNQSLLELHKLATDKNDPHLCDFIETHYLNEQV
KAIKELGDHVTNLRKMGAPESGLAEYLFDKHTLGDSDNES

The cDNA sequence for the cysteine-free PRM1-FTH1 fusion (BamHI and XhoI restriction sites are in bold).

(SEQ ID NO. 14)
GGATCCGCACGTTATCGTAGCAGCCGTAGCCAGAGCCGTAGTCGTTATTA
TCGTCAGCGTCAGCGTAGCCGTCGTCGGCGTCGTCGTAGCAGTCAGACCC
GTCGTCGTGCAATGCGTAGCTCACGTCCGCGTTATCGTCCGCGTAGTCGT
CGCCATAAGCTTGGTAGCACCACCGCAAGCACCAGCCAGGTTCGTCAGAA
TTATCATCAGGATAGCGAAGCAGCAATTAACCGTCAGATTAATCTGGAAC
TGTATGCCAGCTATGTTTATCTGAGCATGAGCTATTATTTCGATCGTGAT
GATGTTGCCCTGAAAAACTTCGCAAAATACTTTCTGCATCAGAGCCATGA
AGAACGTGAACATGCAGAAAAACTGATGAAACTGCAGAATCAGCGTGGTG
GTCGTATCTTTCTGCAGGATATTCAGAAACCGGATTGTGATGATTGGGAA
AGCGGTCTGAATGCAATGGAATGTGCACTGCATCTGGATAAAAATGTTAA
TCAGAGCCTGCTGGAACTGCATAAACTGGCAACCGATAAAAACGATCCGC
ATCTGTGTGATTTTATCGAAACCCATTATCTGAACGAACAGGTGAAAGCC
ATTAAAGAACTGGGTGATCATGTTACCAATCTGCGTAAAATGGGTGCACC
GGAAAGTGGTCTGGCAGAATACCTGTTTGATAAACACACCCTGGGTGATA
GCGATAACGAAAGCTAACTCGAG.

By virtue of having NdeI and XhoI restriction sites at the 5′ and 3′ ends, respectively, in the cDNA fragments, the recombinant DNA can be subsequently ligated into a number of T7 promotor-driven pET expression vectors. Thus, selection of optimal expression vector, fusion type, and expression conditions can be readily evaluated.

A second fusion cDNA construct was designed to fuse human PRM1 with the tetramerization domain of human p73 (p73tet); the amino acid sequence for p73tet was obtained from the NCBI Genbank database (Accession 2WQI_C GI:260656126). The resulting amino acid sequence also included (SEQ ID NO. 14), as before, the residues glycine and serine at the N-terminus due to the BamHI restriction site added to the 5′ end of the cDNA sequence; an ochre stop signal, followed by XhoI restriction site added to the 3′ end of the cDNA sequence. Amino acid sequence for human cysteine-free PRM1-p73tet fusion is G S A R Y R S S R S Q S R S R Y Y R Q R Q R S R R R R R R S S Q T R R R A M R S S R P R Y R P R S R R H K L G N G S D E D T Y Y L Q V R G R E N F E I L M K L K E S L E L M E L V P Q P L V D S Y R Q Q Q Q L L Q R P (SEQ ID NO. 15).

The cDNA sequence for the cysteine-free PRM1-p73tet fusion (BamHI and XhoI restriction sites are in bold). These constructs are as follows:

(SEQ ID NO. 16)
GGATCCCACGTTATCGTAGCAGCCGTAGCCAGAGCCGTAGTCGTTATTAT
CGTCAGCGTCAGCGTAGCCGTCGTCGGCGTCGTCGTAGCAGTCAGACCCG
TCGTCGTGCAATGCGTAGCTCACGTCCGCGTTATCGTCCGCGTAGTCGTC
GCCATAAGCTTGGTAATGGTAGTGATGAAGATACCTACTATCTGCAGGTT
CGTGGTCGTGAAAATTTTGAGATTCTGATGAAACTGAAAGAAAGCCTGGA
ACTGATGGAACTGGTTCCGCAGCCGCTGGTTGATAGTTATCGCCAGCAGC
AGCAACTGCTGCAGCGTCCGTAACTCGAG

### Example 8

Creating the final scFv fusion constructs. The resulting constructs (scFv, PRM1, PRM1-FTH1, and PRM1-p73tet) are designed for direct ligation to create 3 different scFv fusions:

scFv-PRM1(cysteine-free protamine 1) (SEQ ID NO. 10): a monovalent fusion capable of sequestering siRNA and binding to cells that display CD38;

scFv-PRM1-FTH1 (cysteine-free protamine 1) (SEQ ID NO. 12): a polyvalent fusion capable of sequestering siRNA and binding to cells that display CD38; and

scFv-PRM1-p73tet (cysteine-free protamine 1) (SEQ ID NO. 15): a tetravalent fusion capable of sequestering siRNA and binding to cells that display CD38.

### Example 9

Creating scFv fusion constructs with truncated human protamine 1. As the new polyvalent scFv fusions contained multiple potential siRNA binding sites, 2 new fusions were designed to minimize possibility of non-specific binding of cellular nucleic acids to the protamine domain during expression. The new constructs have a new protamine motif (Prm1t) formed by the first 30 amino acids of the cysteine-free protamine design (SEQ ID NO. 10). The amino and cDNA sequences for the human Prm1t-p73tet fusion are shown in (SEQ ID NO. 16) and (SEQ ID NO. 17), respectively.

Amino acid sequence for human Prm1t-p73tet fusion (linkers are in bold) is as follows:

(SEQ ID NO. 16)
GSARYRSSRSQSRSRYYRQRQRSRRRRRRSSQKLGNGSDEDTYYLQVRGR
ENFEILMKLKESLELMELVPQPLVDSYRQQQQLLQRP

The cDNA sequence for the Prm1t-p73tet fusion (BamHI, HindIII and XhoI restriction sites are in bold) is as follows:

(SEQ ID NO. 17)
GGATCCGCACGTTATCGTAGCAGCCGTAGCCAGAGCCGTAGTCGTTATTA
TCGTCAGCGTCAGCGTAGCCGTCGTCGGCGTCGTCGTAGCAGTCAGaagc
ttGGTAATGGTAGTGATGAAGATACCTACTATCTGCAGGTTCGTGGTCGT
GAAAATTTTGAGATTCTGATGAAACTGAAAGAAAGCCTGGAACTGATGGA
ACTGGTTCCGCAGCCGCTGGTTGATAGTTATCGCCAGCAGCAGCAACTGC
TGCAGCGTCCGTAACTCGAG

The amino and cDNA sequences for the human Prm1t-Fth1 fusion are shown in (SEQ ID NO. 18) and (SEQ ID NO. 19), respectively.

The amino acid sequence for the cysteine-free Prm1t-Fth1 fusion (BamHI, HindIII and XhoI restriction sites are in bold) is as follows:

(SEQ ID NO. 18)
GSARYRSSRSQSRSRYYRQRQRSRRRRRRSSQKLTTASTSQVRQNYHQDS
EAAINRQINLELYASYVYLSMSYYFDRDDVALKNFAKYFLHQSHEERAHA
EKLMKLQNQRGGRIFLQDIQKPDRDDWESGLNAMEAALQLDKNVNQSLLE
LHKLATDKNDPHLCDFIETHYLNQQVKAIKQLGDHVTNLRKMGAPESGLA
EYLFDKHTLGDSDNES

cDNA sequence for the cysteine-free Prm1-Fth1 fusion (BamHI, HindIII and XhoI restriction sites are in bold) is as follows:

(SEQ ID NO. 19)
GGATCCGCACGTTATCGTAGCAGCCGTAGCCAGAGCCGTAGTCGTTATTA
TCGTCAGCGTCAGCGTAGCCGTCGTCGGCGTCGTCGTAGCAGTCAGAAGC
TTACCACCGCGTCTACCTCTCAGGTTCGTCAGAACTACCACCAGGACTCT
GAAGCGGCGATCAACCGTCAGATCAACCTGGAACTGTACGCGTCTTACGT
TTACCTGTCTATGTCTTACTACTTCGACCGTGACGACGTTGCGCTGAAAA
ACTTCGCGAAATACTTCCTGCACCAGTCTCACGAAGAACGTGCACACGCG
GAAAAACTGATGAAACTGCAGAACCAGCGTGGTGGTCGTATCTTCCTGCA
GGACATCCAAAAACCGGACCGTGACGACTGGGAATCTGGTCTGAACGCGA
TGGAAGCAGCGCTGCAGCTGGATAAAAACGTTAACCAGTCTCTGCTGGAA
CTGCACAAACTGGCGACCGACAAAAACGACCCGCACCTGTGCGACTTCAT
CGAAACCCACTACCTGAACCAGCAGGTTAAAGCGATCAAACAGCTGGGTG
ACCACGTTACCAACCTGCGTAAAATGGGTGCGCCGGAATCTGGTCTGGCG
GAATACCTGTTCGACAAACACACCCTGGGTGACTCTGACAACGAATCTTA
ACTCGAG

Complete cDNA and amino acid sequences for scFv-Prm1t-p73tet are provided in SEQ ID NO. 20 and SEQ ID NO. 21, respectively.

The scFv-Prm1t-p73tet fusion (cDNA; internal restriction sites and stop in bold) is as follows:

(SEQ ID NO. 20)
ATGGCCGATATTGTTATGACCCAGAGCCAGAAAATCATGCCGACCAGCGT
TGGTGATCGTGTTAGCGTTACCTGTAAAGCAAGCCAGAATGTTGATACCA
ATGTTGCATGGTATCAGCAGAAACCGGGTCAGAGCCCGAAAGCACTGATT
TATAGCGCAAGCTATCGTTATAGCGGTGTTCCGGATCGTTTTACCGGTAG
CGGTAGCGGCACCGATTTTACCCTGACCATTACCAATGTGCAGAGCGAAG
ATCTGGCAGAATATTTCTGTCAGCAGTATGATAGTTATCCGCTGACCTTT
GGTGCAGGTACAAAACTGGATCTGAAACGCGGTGGTGGTGGTTCAGGTGG
TGGTAGCAGTGGTGGCGGTGGTAGCGAAGTTAAACTGATTGAAGCAGGCG
GTGGTCTGGTGCAGCCAGGTGGTAGCCTGAAACTGAGCTGTGCAGCAAGC
GGTTTTGATTTTAGCCGTAGCTGGATGAATTGGGTTCGTCAGGCACCGGG
TAAAGGTCTGGAATGGATTGGTGAAATTAATCCGGATAGCAGCACCATTA
ACTATACCACCAGTCTGAAAGACAAATTTATCATCAGCCGTGACAATGCC
AAAAACACCCTGTATCTGCAAATGACCAAAGTTCGTAGCGAAGATACCGC
ACTGTATTATTGTGCACGTTATGGTAATTGGTTTCCGTATTGGGGTCAGG
GCACCCTGGTTACCGTTAGCGCAGGATCCGCACGTTATCGTAGCAGCCGT
AGCCAGAGCCGTAGTCGTTATTATCGTCAGCGTCAGCGTAGCCGTCGTCG
GCGTCGTCGTAGCAGTCAGAAGCTTGGTAATGGTAGTGATGAAGATACCT
ACTATCTGCAGGTTCGTGGTCGTGAAAATTTTGAGATTCTGATGAAACTG
AAAGAAAGCCTGGAACTGATGGAACTGGTTCCGCAGCCGCTGGTTGATAG
TTATCGCCAGCAGCAGCAACTGCTGCAGCGTCCGTAA

The scFv-Prm1t-p73tet fusion (protein, 328 AA, MW=˜36600) is as follows:

(SEQ ID NO. 21)
MADIVMTQSQKIMPTSVGDRVSVTCKASQNVDTNVAWYQQKPGQSPKALIYSASYRYSG
VPDRFTGSGSGTDFTLTITNVQSEDLAEYFCQQYDSYPLTFGAGTKLDLKRGGGGSGGGS
SGGGGSEVKLIEAGGGLVQPGGSLKLSCAASGFDFSRSWMNWVRQAPGKGLEWIGEINP
DSSTINYTTSLKDKFIISRDNAKNTLYLQMTKVRSEDTALYYCARYGNWFPYWGQGTLVT
VSAGSARYRSSRSQSRSRYYRQRQRSRRRRRRSSQKLGNGSDEDTYYLQVRGRENFEILM
KLKESLELMELVPQPLVDSYRQQQQLLQRP

Complete cDNA and amino acid sequences for scFv-Prm1t-Fth1 are provided in SEQ ID NO. 22 and SEQ ID NO. 23, respectively.

The scFv-Prm1t-Fth1 fusion (cDNA; internal restriction sites and stop in bold) as follows:

(SEQ ID NO. 22)
ATGGCCGATATTGTTATGACCCAGAGCCAGAAAATCATGCCGACCAGCGTTGGTGATC
GTGTTAGCGTTACCTGTAAAGCAAGCCAGAATGTTGATACCAATGTTGCATGGTATCA
GCAGAAACCGGGTCAGAGCCCGAAAGCACTGATTTATAGCGCAAGCTATCGTTATAG
CGGTGTTCCGGATCGTTTTACCGGTAGCGGTAGCGGCACCGATTTTACCCTGACCATT
ACCAATGTGCAGAGCGAAGATCTGGCAGAATATTTCTGTCAGCAGTATGATAGTTATC
CGCTGACCTTTGGTGCAGGTACAAAACTGGATCTGAAACGCGGTGGTGGTGGTTCAGG
TGGTGGTAGCAGTGGTGGCGGTGGTAGCGAAGTTAAACTGATTGAAGCAGGCGGTGG
TCTGGTGCAGCCAGGTGGTAGCCTGAAACTGAGCTGTGCAGCAAGCGGTTTTGATTTT
AGCCGTAGCTGGATGAATTGGGTTCGTCAGGCACCGGGTAAAGGTCTGGAATGGATT
GGTGAAATTAATCCGGATAGCAGCACCATTAACTATACCACCAGTCTGAAAGACAAA
TTTATCATCAGCCGTGACAATGCCAAAAACACCCTGTATCTGCAAATGACCAAAGTTC
GTAGCGAAGATACCGCACTGTATTATTGTGCACGTTATGGTAATTGGTTTCCGTATTG
GGGTCAGGGCACCCTGGTTACCGTTAGCGCAGGATCCGCACGTTATCGTAGCAGCCG
TAGCCAGAGCCGTAGTCGTTATTATCGTCAGCGTCAGCGTAGCCGTCGTCGGCGTCGT
CGTAGCAGTCAGAAGCTTACCACCGCGTCTACCTCTCAGGTTCGTCAGAACTACCACC
AGGACTCTGAAGCGGCGATCAACCGTCAGATCAACCTGGAACTGTACGCGTCTTACGT
TTACCTGTCTATGTCTTACTACTTCGACCGTGACGACGTTGCGCTGAAAAACTTCGCGA
AATACTTCCTGCACCAGTCTCACGAAGAACGTGCACACGCGGAAAAACTGATGAAAC
TGCAGAACCAGCGTGGTGGTCGTATCTTCCTGCAGGACATCCAAAAACCGGACCGTG
ACGACTGGGAATCTGGTCTGAACGCGATGGAAGCAGCGCTGCAGCTGGATAAAAACG
TTAACCAGTCTCTGCTGGAACTGCACAAACTGGCGACCGACAAAAACGACCCGCACC
TGTGCGACTTCATCGAAACCCACTACCTGAACCAGCAGGTTAAAGCGATCAAACAGCT
GGGTGACCACGTTACCAACCTGCGTAAAATGGGTGCGCCGGAATCTGGTCTGGCGGA
ATACCTGTTCGACAAACACACCCTGGGTGACTCTGACAACGAATCTTAA

The scFv-Prm1t-Fth1 fusion (protein, 457 AA, MW=51,336) is as follows:

(SEQ ID NO. 23)
MADIVMTQSQKIMPTSVGDRVSVTCKASQNVDTNVAWYQQKPGQSPKALIYSASYRYSG
VPDRFTGSGSGTDFTLTITNVQSEDLAEYFCQQYDSYPLTFGAGTKLDLKRGGGGSGGGS
SGGGGSEVKLIEAGGGLVQPGGSLKLSCAASGFDFSRSWMNWVRQAPGKGLEWIGEINP
DSSTINYTTSLKDKFIISRDNAKNTLYLQMTKVRSEDTALYYCARYGNWFPYWGQGTLVT
VSAGSARYRSSRSQSRSRYYRQRQRSRRRRRRSSQKLTTASTSQVRQNYHQDSEAAINRQI
NLELYASYVYLSMSYYFDRDDVALKNFAKYFLHQSHEERAHAEKLMKLQNQRGGRIFLQ
DIQKPDRDDWESGLNAMEAALQLDKNVNQSLLELHKLATDKNDPHLCDFIETHYLNQQV
KAIKQLGDHVTNLRKMGAPESGLAEYLFDKHTLGDSDNES

### REFERENCES

Almeida J, Orfao A, Mateo G, Ocqueteau M, Garcia-Sanz R, Moro M J, Hernandez J, Ortega F, Borrego D, Barez A, Mejida M, San Miguel J F. Immunophenotypic and DNA content characteristics of plasma cells in multiple myeloma and monoclonal gammopathy of undetermined significance. Path Biol 1999; 47:119-127.
Anderson D C, Nichols E, Manger R, Woodle D, Barry M, Fritzberg A R. Tumor cell retention of antibody Fab fragments is enhanced by an attached HIV TAT protein-derived peptide. Biochem Biophys Res Commun 1993; 194:876-884.
Bauer S, Abdgawad M, Gunnarsson L, Segelmark M, Tapper H, and Hellmark T. Proteinase 3 and CD177 are expressed on the plasma membrane of the same subset of neutrophils. J. Leukoc. Biol. 2007; 81:458-464
Becker J M. Allergic Rhinitis, in In eMedicine, eds: Park C L, Mary L Windle M L, Georgitis J W, Pallares D, MD, Ballow M. 2004.
Brake M, Somers D. IgA Nephropathy in eMedicine, eds: Sondheimer J H, Talavera, F, Thomas C, Schmidt R J, Vecihi Batuman V. 2003.
Caron N J, Quenneville S P, Tremblay J P. Endosome disruption enhances functional nuclear delivery of Tat-fusion proteins. Biochem Biophys Res Commun 2004; 319:12-20.
CellSensor CRE-bla Jurkat Cell-based Assay Protocol, Catalogue number K1134 (K1079), Invitrogen Corporation, Carlsbad, Calif.
Chiu Y- L, Ali A, Chu C-y, Cao H, Rana T M. Visualizing a correlation between siRNA localization, cellular uptake, and RNAi in living cells. Chem Biol 2004; 11:1165-1175.
Diallo M, Arenz C, Schmitz K, Sandhoff K, Scheppers U. Long endogenous dsRNAs can induce complete gene silencing in mammalian cells and primary cultures. Oligonucleotides 2003; 13:381-392.
Funaro A, Reinis M, Trubiani O, Santi S, Di Primio R, Malavasi F. CD38 functions are regulated through an internalization step. J Immunol 1998; 160:2238-2247.
Futaki S, Goto S, Sugiura Y. Membrane permeability commonly shared among arginine-rich peptides. J Mol Recognit 2003; 16:260-264.
Grethlein S. Multiple Myeloma. In eMedicine, eds: Krishnan K, Talavera F, Guthrie T H, McKenna Rajalaxmi, Besa E C 2003.
He D, Yang H, Lin Q, Huang H. Arg9-peptide facilitates the internalization of an anti-CEA imunotoxin and potentiates its specific cytotoxity to target cells. Int J Biochem Cell Biol 2005; 37:192-205.
Hermanson G T. Bioconjugate Techniques. Academic Press, San Diego, Calif. 1996.
Hutvagner G, Zamore P D. A microRNA in a multiple-turnover RNAi enzyme complex. Nature 2002; 297:2056-2060.
Hutvagner G, Zamore P D. RNAi: nature abhors a double-strand. Curr Opinion in Genetics and Development 2002; 12:225-232.
Jacobs B L, Imani F. Histone proteins inhibit activation of the interferon-induced protein kinase by binding to double-stranded RNA. J Interferon Res 1988; 8:821-830.
Jo D, Nashabi A, Doxee C, Lin Q, Unutmaz D, Chen J, Ruley H E. Epigenetic regulation of gene structure and function with a cell-permeable Cre recombinase. Nature Biotechnology 2001; 19:929-933.
Jones S, Daley T A, Luscombe N M, Berman H M, Thornton J M. Protein-RNA interactions: a structural analysis. Nucl Acids Res 2001; 29:943-954.
Kabat E A, Wu T T, Perry H M, Gottesman K S, Foeller C. Sequences of Proteins of Immunological Interest. Fifth Edition. Tabulation and Analysis of Amino Acid and Nucleic Acid Sequences of Precursors, V-Regions, C-Regions, J-Chain, T-Cell Receptors for Antigen, T-Cell Surface Antigens, β₂-Microglobulins, Major Histocompatibility Antigens, Thy-1, Complement, C-Reactive Protein, Thymopoietin, Integrins, Post-gamma Globulin, α₂-Macroglobulins, and other Related Proteins. 1991. NIH Publication Number 91-3242.
Kratzmeier M, Albig W, Hanecke K, Doenecke D. Rapid dephosphorylation of H1 histones after apoptosis induction. J Biol Chem. 2000; 275:30478-30486.
Lee C- H, Hoyer-Fender S, Engel W. The nucleotide sequence of a human protamine 1 cDNA. Nucleic Acids Research 1987; 15:7639.
Mie M, Takahashi F, Funabashi H, Yanagida Y, Aizawa M, Kobatake E. Intracellular delivery of antibodies using TAT fusion protein A. Biochem Biophys Res Commun 2003; 310:730-734.
Miller V M, Xia H, Marrs G L, Gouvion C M, Lee G, Davidson B L, Paulson H L. Allele-specific silencing of dominant disease genes. Proc Natl Acad Sci USA 2003; 100:7195-7200.
Miyagishi M, Taira K. Strategies for generation of an siRNA expression library directed against the human genome. Oligonucleotides 2003; 13:325-333.
Muratovska A, Eccles M R. Conjugate for efficient delivery of short interfering RNA (siRNA) into mammalian cells. FEBS Letters 2004; 558:63-68.
Myers J W, Jones J T, Meyer T, Ferrell J E Jr. Recombinant Dicer efficiently converts large dsRNAs into siRNAs suitable for gene silencing. Nature Biotechnology 2003; 21:324-328.
Parrish S, Fire A. Distinct roles for RDE-1 and RDE-4 during RNA interference in Caenorhabditis elegans. RNA 2001; 7:1397-1402.
Peitz M, Pfannkuche K, Rajewsky K, Edenhofer F. Ability of the hydrophobic FGF and basic TAT peptides to promote cellular uptake of the recombinant Cre recombinase: A tool for efficient genetic engineering of mammalian genomes. Proc Natl Acad Sci USAS 2002; 99:4489-4494.
Pfister M, Ogilvie A, da Silva C P, Grahnert A, Guse A H, Hauschildt S. NAD degradation and regulation of CD38 expression by human monocytes/macrophages. Eur J Biochem 2001; 268:5601-5608.
Provost P, Dishart D, Doucer J, Frendewey D, Samuelsson B, Radmark O. Ribonuclease activity and RNA binding of recombinant human Dicer. EMBO J 2002; 21:5864-5874.
Scott L M, Tong W, Levine R L, Scott M A, Beer P A, Stratton M R, Futreal P A, Erber W N, McMullin M F, Harrison C N, Warren A J, Gilliland D G, Lodish H F, Green A R. JAK2 exon 12 mutations in polycythemia vera and idiopathic erythrocytosis. N Engl J Med. 2007; 356:459-68.
St. Johnston D, Brown N H, Gall J G, Jantsch M. A conserved double-stranded RNA-binding domain. Proc Natl Acad Sci USA 1992; 89:10979-10983.
Saunders L A, Barber G N. The dsRNA binding protein family: critical roles, diverse cellular functions. FASEB J 2003; 17:961-983.
Siraganian R P. Biochemical events in basophil or mast cell activation and mediator release. Chapter 16 pp 204-227 in Allergy Principles and Practice, 5^{th} edition, eds E Middleton, Jr, C E Reed, E F Ellis, N F Adkinson, Jr, J W Yunginger W W Busse. Mosby, St. Louis, 1998.
Song E, Zhu P, Lee S- K, Chowdury D, Kussman S, Dykxhoorn D M, Feng Y, Palliser D, Weiner D B, Shankar P, Marasco W A, Lieberman J. Antibody mediated in vivo delivery of small interfering RNAs via cell-surface receptors. Nature Biotechnology (epublication): 22 May 2005; doi:10.1038/nbt1101; (paper publication): 2005; 23:709-717.
Soomets U, Lindgren M, Gallet X, Hallbrink M, Elmquist A, Balaspiri L, Zorka M, Pooga M, Brasseur R, Langel U. Deletion analogues of transportan. Biochem Biophys Acta 2000; 1467:165-176.
Stura E A, Fieser G G, Wilson I A. Crystallization of antibodies and antibody-antigen complexes. Immunomethods 1993; 3:164-179.
Tabara H, Yigit E, Siomi H, Mello C C. The dsRNA binding protein RDE-4 interacts with RDE-1, DCR-1 and a DexH-Box helicase to direct RNAi in C. elegeans. Cell 2002; 109:861-871.
Temerinac S., Klippel S, Strunck E, Roder S, Lübbert M, Lange S, Azemar M, Meinhardt G, Schaefer H, and Pahl H, Cloning of PRV-1, a novel member of the uPAR receptor superfamily, which is overexpressed in polycythemia rubra vera. Blood 2000; 95: 2569-2576.
van Koningsbruggen S, de Haard H, de Kievit P, Dirks R W, van Remoortere A, Groot A J, van Engelen B G, den Dunnen J T, Verrips C T, Frants R R, van der Maarel S M. Llama-derived phage display antibodies in the dissection of the human disease oculopharyngeal muscular dystrophy. J Immunol Methods 2003; 279: 149-161.
Warrant R W, Kim S- H. α-Helix-double helix interaction shown in the structure of a protamine-transfer RNA complex and a nucleoprotamine model. Nature 1978; 271:130-135.
Waterhouse P M, Wang M- B, Lough T. Gene silencing as an adaptive defense against viruses. Nature 2001; 411:834-842.
Yaneva J, Leuba S H, van Holde K, Zlatanova J. The major chromatin protein histone H1 binds preferentially to cis-platinum-damaged DNA. Proc Natl Acad Sci USA 1997; 94:13448-13451.
Yoshikawa Y, Velichko Y S, Ichiba Y, Yoshikawa K. Self-assembled pearling structure of long duplex DNA with histone H1. Eur J Biochem 2001; 268:2593-2599.
Zhang H, Kolb F A, Brondini V, Billy E, Filipowicz W. Human Dicer preferentially cleaves dsRNAs at their termini without a requirement for ATP. EMBO J 2002; 21:5875-5885.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A fusion protein comprising:
a cell surface receptor specific synthetic single chain variable domain (scFv) immunoglobulin fragment vehicle specific to a cell surface receptor of a cell and having a cell surface receptor specific binding site;
an RNA binding protein fused to said scFv; and
a double-stranded RNA or to a small hairpin RNA sequence complementary to a nucleotide sequence of a target gene in the cell and comprising a small interfering RNA operative to suppress production of a cellular protein, said double-stranded RNA or said small hairpin RNA sequence adsorbing said RNA binding protein;
said scFv induces internalization into said cell of the fusion protein subsequent to the binding of said scFV to the cell surface receptor of the target cell.

2. The fusion protein of claim 1 wherein said RNA binding protein is selected from the group consisting of: histone, protamine, cysteine-less human protamine 1 fused with the heavy chain of human ferritin, RDE4 and PKR (Accession number in parenthesis) (AAA36409, AAA61926, Q03963), TRBP (P97473, AAA36765), PACT (AAC25672, AAA49947, NP_{—}609646), Staufen (AAD17531, AAF98119, AAD17529, P25159), NFAR1 (AF167569), NFAR2 (AF167570, AAF31446, AAC71052, AAA19960, AAA19961, AAG22859), SPNR (AAK20832, AAF59924, A57284), RHA (CAA71668, AAC05725, AAF57297), NREBP (AAK07692, AAF23120, AAF54409, T33856), kanadaptin (AAK29177, AAB88191, AAF55582, NP_{—}499172, NP_{—}198700, BAB19354), HYL1 (NP_{—}563850), hyponastic leaves (CAC05659, BAB00641), ADAR1 (AAB97118, P55266, AAK16102, AAB51687, AF051275), ADAR2 P78563, P51400, AAK17102, AAF63702), ADAR3 (AAF78094, AAB41862, AAF76894), TENR (XP_{—}059592, CAA59168), RNaseIII (AAF80558, AAF59169, Z81070Q02555/S55784, P05797), and Dicer (BAA78691, AF408401, AAF56056, S44849, AAF03534, Q9884), RDE-4 (AY071926), F1120399 (NP_{—}060273, BAB26260), CG1434 (AAF48360, EAA12065, CAA21662), CG13139 (XP_{—}059208, XP_{—}143416, XP_{—}110450, AAF52926, EEA14824), DGCRK6 (BAB83032, XP_{—}110167) CG1800 (AAF57175, EAA08039), F1120036 (AAH22270, XP_{—}134159), MRP-L45 (BAB14234, XP_{—}129893), CG2109 (AAF52025), CG12493 (NP_{—}647927), CG10630 (AAF50777), CG17686 (AAD50502), T22A3.5 (CAB03384) and nameless Accession number EAA14308.

3. The fusion protein of claim 1 wherein said scFv is monomeric.

4. The fusion protein of claim 1 wherein said scFv is tetravalent.

5. The fusion protein of claim 1 wherein said scFv is polyvalent.

6. The fusion protein of claim 1 wherein said double-stranded RNA is complementary to a cellular nucleotide sequence for a cell binding said ligand.

7. The fusion protein of claim 1 wherein the ligand and RNA binding protein are conjugated in vitro.

8. The fusion protein of claim 1 further comprising an internalization moiety having a bond to said scFv.

9. The fusion protein of claim 1 wherein said internalization moiety has a bond to said RNA binding protein.

10. The fusion protein of claim 9 wherein said internalization moiety is selected from the group of membrane-permeable arginine-rich peptides, pentratin, transportan, and transportan deletion analogs.

11. The fusion protein of claim 1 wherein said scFv is an anti-CD177 synthetic single chain variable domain (scFv) immunoglobulin fragment vehicle and said double-stranded RNA is complementary to a portion of a malignant cell genome.

12. The fusion protein of claim 1 wherein said small interfering RNA sequence is complementary to a JAK2 sequence.

13. The fusion protein of claim 1 wherein said scFv is an anti-CD177 synthetic single chain variable domain (scFv) immunoglobulin fragment vehicle and said double-stranded RNA is coding for an anti-JAK2 small interfering RNA.

14. The protein of claim 13 wherein said internalization moiety is selected from the group of membrane-permeable arginine-rich peptides, pentratin, transportan, and transportan deletion analogs.

15. The fusion protein of claim 1 wherein said RNA binding protein is free of cysteine residues.

16. The fusion protein of claim 1 having an amino acid sequence of one of SEQ ID NO 10, 12, or 15.

17. The fusion protein of claim 1 having an amino acid sequence of one of SEQ ID NO 18, 21, or 23.

18. A process for suppressing cellular production of a protein comprising: exposing a cell having a cell surface receptor to the fusion protein of claim 1.


SEQUENCE LISTING (24 sequences)

SEQ ID NO: 1 - protein, 15 aa, Homo sapiens
RSRRRRRRSCQTRRR

SEQ ID NO: 2 - DNA, 795 bp, Homo sapiens
atggacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 3 - DNA, 19 bp, Artificial Sequence
  Synthethic
ccacgatgcctgtagcaat

SEQ ID NO: 4 - RNA, 21 nt, Artificial Sequence
  Synthetic
cgccaagaacuuggucuauuu

SEQ ID NO: 5 - RNA, 21 nt, Artificial Sequence
  Synthetic
uugcgguucuugaaccagaua

SEQ ID NO: 6 - DNA, 14 bp, Artificial Sequence
  Synthetic
ggggsgggsggggs

SEQ ID NO: 7 - DNA, 732 bp, Artificial Sequence
  Synthetic
catatggccgatattgttatgacccagagccagaaaatcatgccgaccagcgttggtgatcgtgttagcgttacctgtaaagcaagccagaatgttgataccaatgttgcatggtatcagcagaaaccgggtcagagcccgaaagcactgatttatagcgcaagctatcgttatagcggtgttccggatcgttttaccggtagcggtagcggcaccgattttaccctgaccattaccaatgtgcagagcgaagatctggcagaatatttctgtcagcagtatgatagttatccgctgacctttggtgcaggtacaaaactggatctgaaacgcggtggtggtggttcaggtggtggtagcagtggtggcggtggtagcgaagttaaactgattgaagcaggcggtggtctggtgcagccaggtggtagcctgaaactgagctgtgcagcaagcggttttgattttagccgtagctggatgaattgggttcgtcaggcaccgggtaaaggtctggaatggattggtgaaattaatccggatagcagcaccattaactataccaccagtctgaaagacaaatttatcatcagccgtgacaatgccaaaaacaccctgtatctgcaaatgaccaaagttcgtagcgaagataccgcactgtattattgtgcacgttatggtaattggtttccgtattggggtcagggcaccctggttaccgttagcgcaggatcc

SEQ ID NO: 8 - protein, 243 aa, Artificial Sequence
  Synthetic
MADIVMTQSQKIMPTSVGDRVSVTCKASQNVDTNVAWYQQKPGQSPKALIYSASYRYSGVPDRFTGSGSGTDFTLTITNVQSEDLAEYFCQQYDSYPLTFGAGTKLDLKRGGGGSGGGSSSGGGGSEVKLIEAGGGLVQPGGSLKLSCAASGFDFSRSWMNWVRQAPGKGLEWIGEINPDSTINYTTSLKDKFIISRDNAKNTLYLQMTKVRSEDTALYYCARYGNWFPYWGQGTLVTVSAGS

SEQ ID NO: 9 - protein, 51 aa, Homo sapiens
MARYRCCRSQSRSRYYRQRQRSRRRRRRSCQTRRRAMRCCRPRYRPRCRRH

SEQ ID NO: 10 - protein, 52 aa, Artificial Sequence
  Synthetic
GSARYRSSRSQSRSRYYRQRQRSRRRRRRSSQTRRRAMRSSRPRYRPRSRRH

SEQ ID NO: 11 - protein, 183 aa, Homo sapiens
MTTASTSQVRQNYHQDSEAAINRQINLELYASYVYLSMSYYFDRDDVALKNFAKYFLHQSHEEREHAEKLMKLQNQRGGRIFLQDIQKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 12 - protein, 238 aa, Homo sapiens
GSARYRSSRSQSRSRYYRQRQRSRRRRRRSSQTRRRAMRSSRPRYRPRSRRHKLGSTTASTSQVRQNYHQDSEAAINRQINLELYASYVYLSMSYYFDRDDVALKNFAKYFLHQSHEEREHAEKLMKLQNQRGGRIFLQDIQKPDCDDWESGLNAMECALHLDKNVNQSLLELHKLATDKNDPHLCDFIETHYLNEQVKAIKELGDHVTNLRKMGAPESGLAEYLFDKHTLGDSDNES

SEQ ID NO: 13 - protein, 238 aa, Homo sapiens; identical to SEQ ID NO: 12

SEQ ID NO: 14 - DNA, 723 bp, Homo sapiens
ggatccgcacgttatcgtagcagccgtagccagagccgtagtcgttattatcgtcagcgtcagcgtagccgtcgtcggcgtcgtcgtagcagtcagacccgtcgtcgtgcaatgcgtagctcacgtccgcgttatcgtccgcgtagtcgtcgccataagcttggtagcaccaccgcaagcaccagccaggttcgtcagaattatcatcaggatagcgaagcagcaattaaccgtcagattaatctggaactgtatgccagctatgtttatctgagcatgagctattatttcgatcgtgatgatgttgccctgaaaaacttcgcaaaatactttctgcatcagagccatgaagaacgtgaacatgcagaaaaactgatgaaactgcagaatcagcgtggtggtcgtatctttctgcaggatattcagaaaccggattgtgatgattgggaaagcggtctgaatgcaatggaatgtgcactgcatctggataaaaatgttaatcagagcctgctggaactgcataaactggcaaccgataaaaacgatccgcatctgtgtgattttatcgaaacccattatctgaacgaacaggtgaaagccattaaagaactgggtgatcatgttaccaatctgcgtaaaatgggtgcaccggaaagtggtctggcagaatacctgtttgataaacacaccctgggtgatagcgataacgaaagctaactcgag

SEQ ID NO: 15 - protein, 107 aa, Artificial Sequence
  Synthetic
GSARYRSSRSQSRSRYYRQRQRSRRRRRRSSQTRRRAMRSSRPRYRPRSRRHKLGNGSDEDTYYLQVRGRENFEILMKLKESLELMELVPQPLVDSYRQQQQLLQRP

SEQ ID NO: 16 - DNA, 329 bp, Artificial Sequence
  synthetic
ggatcccacgttatcgtagcagccgtagccagagccgtagtcgttattatcgtcagcgtcagcgtagccgtcgtcggcgtcgtcgtagcagtcagacccgtcgtcgtgcaatgcgtagctcacgtccgcgttatcgtccgcgtagtcgtcgccataagcttggtaatggtagtgatgaagatacctactatctgcaggttcgtggtcgtgaaaattttgagattctgatgaaactgaaagaaagcctggaactgatggaactggttccgcagccgctggttgatagttatcgccagcagcagcaactgctgcagcgtccgtaactcgag

SEQ ID NO: 17 - protein, 87 aa, Artificial Sequence
  synthetic
GSARYRSSRSQSRSRYYRQRQRSRRRRRRSSQKLGNGSDEDTYYLQVRGRENFEILMKLKESLELMELVPQPLVDSYRQQQQLLQRP

SEQ ID NO: 18 - DNA, 270 bp, Artificial Sequence
  Synthetic
ggatccgcacgttatcgtagcagccgtagccagagccgtagtcgttattatcgtcagcgtcagcgtagccgtcgtcggcgtcgtcgtagcagtcagaagcttggtaatggtagtgatgaagatacctactatctgcaggttcgtggtcgtgaaaattttgagattctgatgaaactgaaagaaagcctggaactgatggaactggttccgcagccgctggttgatagttatcgccagcagcagcaactgctgcagcgtccgtaactcgag

SEQ ID NO: 19 - protein, 216 aa, Artificial Sequence
  synthetic
GSARYRSSRSQSRSRYYRQRQRSRRRRRRSSQKLTTASTSQVRQNYHQDSEAAINRQINLELYASYVYLSMSYYFDRDDVALKNFAKYFLHQSHEERAHAEKLMKLQNQRGGRIFLQDIQKPDRDDWESGLNAMEAALQLDKNVNQSLLELHKLATDKNDPHLCDFIETHYLNQQVKAIKQLGDHVTNLRKMGAPESGLAEYLFDKHTLGDSDNES

SEQ ID NO: 20 - DNA, 657 bp, Artificial Sequence
  synthetic
ggatccgcacgttatcgtagcagccgtagccagagccgtagtcgttattatcgtcagcgtcagcgtagccgtcgtcggcgtcgtcgtagcagtcagaagcttaccaccgcgtctacctctcaggttcgtcagaactaccaccaggactctgaagcggcgatcaaccgtcagatcaacctggaactgtacgcgtcttacgtttacctgtctatgtcttactacttcgaccgtgacgacgttgcgctgaaaaacttcgcgaaatacttcctgcaccagtctcacgaagaacgtgcacacgcggaaaaactgatgaaactgcagaaccagcgtggtggtcgtatcttcctgcaggacatccaaaaaccggaccgtgacgactgggaatctggtctgaacgcgatggaagcagcgctgcagctggataaaaacgttaaccagtctctgctggaactgcacaaactggcgaccgacaaaaacgacccgcacctgtgcgacttcatcgaaacccactacctgaaccagcaggttaaagcgatcaaacagctgggtgaccacgttaccaacctgcgtaaaatgggtgcgccggaatctggtctggcggaatacctgttcgacaaacacaccctgggtgactctgacaacgaatcttaactcgag

SEQ ID NO: 21 - DNA, 987 bp, Homo sapiens
atggccgatattgttatgacccagagccagaaaatcatgccgaccagcgttggtgatcgtgttagcgttacctgtaaagcaagccagaatgttgataccaatgttgcatggtatcagcagaaaccgggtcagagcccgaaagcactgatttatagcgcaagctatcgttatagcggtgttccggatcgttttaccggtagcggtagcggcaccgattttaccctgaccattaccaatgtgcagagcgaagatctggcagaatatttctgtcagcagtatgatagttatccgctgacctttggtgcaggtacaaaactggatctgaaacgcggtggtggtggttcaggtggtggtagcagtggtggcggtggtagcgaagttaaactgattgaagcaggcggtggtctggtgcagccaggtggtagcctgaaactgagctgtgcagcaagcggttttgattttagccgtagctggatgaattgggttcgtcaggcaccgggtaaaggtctggaatggattggtgaaattaatccggatagcagcaccattaactataccaccagtctgaaagacaaatttatcatcagccgtgacaatgccaaaaacaccctgtatctgcaaatgaccaaagttcgtagcgaagataccgcactgtattattgtgcacgttatggtaattggtttccgtattggggtcagggcaccctggttaccgttagcgcaggatccgcacgttatcgtagcagccgtagccagagccgtagtcgttattatcgtcagcgtcagcgtagccgtcgtcggcgtcgtcgtagcagtcagaagcttggtaatggtagtgatgaagatacctactatctgcaggttcgtggtcgtgaaaattttgagattctgatgaaactgaaagaaagcctggaactgatggaactggttccgcagccgctggttgatagttatcgccagcagcagcaactgctgcagcgtccgtaa

SEQ ID NO: 22 - protein, 328 aa, Artificial Sequence
  Synthetic
MADIVMTQSQKIMPTSVGDRVSVTCKASQNVDTNVAWYQQKPGQSPKALIYSASYRYSGVPDRFTGSGSGTDFTLTITNVQSEDLAEYFCQQYDSYPLTFGAGTKLDLKRGGGGSGGGSSGGGGSEVKLIEAGGGLVQPGGSLKLSCAASGFDFSRSWMNWVRQAPGKGLEWIGEINPDSSTINYTTSLKDKFIISRDNAKNTLYLQMTKVRSEDTALYYCARYGNWFPYWGQGTLVTVSAGSARYRSSRSQSRSRYYRQRQRSRRRRRRSSQKLGNGSDEDTYYLQVRGRENFEILMKLKESLELMELVPQPLVDSYRQQQQLLQRP

SEQ ID NO: 23 - DNA, 1374 bp, Homo sapiens
atggccgatattgttatgacccagagccagaaaatcatgccgaccagcgttggtgatcgtgttagcgttacctgtaaagcaagccagaatgttgataccaatgttgcatggtatcagcagaaaccgggtcagagcccgaaagcactgatttatagcgcaagctatcgttatagcggtgttccggatcgttttaccggtagcggtagcggcaccgattttaccctgaccattaccaatgtgcagagcgaagatctggcagaatatttctgtcagcagtatgatagttatccgctgacctttggtgcaggtacaaaactggatctgaaacgcggtggtggtggttcaggtggtggtagcagtggtggcggtggtagcgaagttaaactgattgaagcaggcggtggtctggtgcagccaggtggtagcctgaaactgagctgtgcagcaagcggttttgattttagccgtagctggatgaattgggttcgtcaggcaccgggtaaaggtctggaatggattggtgaaattaatccggatagcagcaccattaactataccaccagtctgaaagacaaatttatcatcagccgtgacaatgccaaaaacaccctgtatctgcaaatgaccaaagttcgtagcgaagataccgcactgtattattgtgcacgttatggtaattggtttccgtattggggtcagggcaccctggttaccgttagcgcaggatccgcacgttatcgtagcagccgtagccagagccgtagtcgttattatcgtcagcgtcagcgtagccgtcgtcggcgtcgtcgtagcagtcagaagcttaccaccgcgtctacctctcaggttcgtcagaactaccaccaggactctgaagcggcgatcaaccgtcagatcaacctggaactgtacgcgtcttacgtttacctgtctatgtcttactacttcgaccgtgacgacgttgcgctgaaaaacttcgcgaaatacttcctgcaccagtctcacgaagaacgtgcacacgcggaaaaactgatgaaactgcagaaccagcgtggtggtcgtatcttcctgcaggacatccaaaaaccggaccgtgacgactgggaatctggtctgaacgcgatggaagcagcgctgcagctggataaaaacgttaaccagtctctgctggaactgcacaaactggcgaccgacaaaaacgacccgcacctgtgcgacttcatcgaaacccactacctgaaccagcaggttaaagcgatcaaacagctgggtgaccacgttaccaacctgcgtaaaatgggtgcgccggaatctggtctggcggaatacctgttcgacaaacacaccctgggtgactctgacaacgaatcttaa

SEQ ID NO: 24 - protein, 457 aa, Artificial Sequence
  Synthetic
MADIVMTQSQKIMPTSVGDRVSVTCKASQNVDTNVAWYQQKPGQSPKALIYSASYRYSGVPDRFTGSGSGTDFTLTITNVQSEDLAEYFCQQYDSYPLTFGAGTKLDLKRGGGGSGGGSSGGGGSEVKLIEAGGGLVQPGGSLKLSCAASGFDFSRSWMNWVRQAPGKGLEWIGEINPDSSTINYTTSLKDKFIISRDNAKNTLYLQMTKVRSEDTALYYCARYGNWFPYWGQGTLVTVSAGSARYRSSRSQSRSRYYRQRQRSRRRRRRSSQKLTTASTSQVRQNYHQDSEAAINRQINLELYASYVYLSMSYYFDRDDVALKNFAKYFLHQSHEERAHAEKLMKLQNQRGGRIFLQDIQKPDRDDWESGLNAMEAALQLDKNVNQSLLELHKLATDKNDPHLCDFIETHYLNQQVKAIKQLGDHVTNLRKMGAPESGLAEYLFDKHTLGDSDNES